(19) United States
(12) Patent Application Publication
(10) Pub. No.: US 2017/0252278 A1
(43) Pub. Date: Sep. 7, 2017

(54) INHIBITION OF MATURATION OF DENTAL BIOFILM AND CARIOGENIC PROPERTIES

(71) Applicant: KONINKLIJKE PHILIPS N.V., EINDHOVEN (NL)

(72) Inventors: MARLEEN MARGA JANUS, EINDHOVEN (NL); BASTIANN PHILIP KROM, EINDHOVEN (NL); WIM CRIELAARD, EINDHOVEN (NL); BART JAN FREDERIK KEIJSER, EINDHOVEN (NL)

(21) Appl. No.: 15/509,537

(22) PCT Filed: Sep. 9, 2015
(86) PCT No.: PCT/EP2015/070551

§ 371 Date: Mar. 8, 2017

(30) Foreign Application Priority Data
Sep. 9, 2014 (EP) 14184146.0

(51) Int. Cl. A61K 8/42; A61Q 11/00; A61B 17/24; A61K 8/02

(52) U.S. Cl. CPC A61K 8/42; A61K 8/0204; A61Q 11/00; A61B 17/244; A61K 2800/87; A61K 2800/28; A61K 2800/48; A61K 2800/524; A61K 2800/92

(57) ABSTRACT

The present invention relates to a small molecule inhibitor for use in the prevention of maturation of dental plaque biofilms. A further application may be the reduction of the risk for diseases associated with functions that derive from matured dental plaque biofilms. The invention also relates to the use of an effective amount of a small molecule inhibitor in a cosmetic product for the prevention of maturation of dental biofilms. The present invention also relates to oral compositions comprising an effective amount of a small molecule inhibitor suitable for the prevention of maturation of dental plaque biofilms. The present invention further relates to a device for application of oral compositions further comprising an oral composition comprising an effective amount of a small molecule inhibitor suitable for the prevention of maturation of dental plaque biofilms.

### FIELD OF THE INVENTION

The invention relates to the inhibition of maturation of dental plaque biofilm and cariogenic properties of dental plaque biofilm as well as to products which inhibit the cariogenic activity of dental plaque biofilms biofilms. The invention further relates to a device using such product.

### BACKGROUND OF THE INVENTION

Dental caries is an infection of bacterial origin, which causes demineralization and destruction of the hard tissues of the teeth. Tooth decay is caused by specific types of bacteria that produce lactic acid in the presence of fermentable carbohydrates such as sucrose, fructose and glucose, e.g. from food debris accumulated on the tooth surface. The mineral content of teeth is sensitive to increases in acidity from the production of lactic acid.

The bacteria most responsible for dental caries are the mutans streptococci, most prominently Streptococcus mutans and Streptococcus sobrinus, and lactobacilli. If left untreated, caries can lead to pain, tooth loss and infection.

As there is no known method to regenerate large amounts of tooth structure when destroyed by caries preventive and prophylactic measures, such as regular oral hygiene and dietary modifications are widely advocated, to avoid dental caries. Despite this, caries remains one of the most common problems to humans throughout the world.

Apart from prevention, oral hygiene and dietary modifications, there has been much attention concerning the microbiological aspects of and in particular to interference with bacterial activity itself. Many methods were studied to either reduce in number the bacteria responsible for the development of caries, or to inhibit the activity of these bacteria leading to the development of caries. However, inhibition of the cariogenic activity of the bacteria poses a problem as the bacteria tend to settle in the mouth in the form of bacterial biofilms—surface-attached bacterial communities embedded in an extracellular matrix of polysaccharides, proteins and DNA. In these dental plaque biofilms, the bacteria are able to withstand host immune responses and are not only more resistant to antibiotics and biocides than if they were in planktonic form, but are also able to better withstand mechanical removal.

Dental plaque bio films forms in several stages, accompanied with different levels of pathogenicity. Young dental plaque bio films (0-8 hour old) are considered normal as they accumulate in every individual. Such young dental plaque biofilms have low acid producing capacity and thus have relatively low cariogenic properties. Maturation of dental plaque biofilms from 8-48 hours result in significant increase in acid producing capacity and increased cariogenic potential of the dental plaque biofilms. Eventually, mature dental plaque biofilms (older than about 48 hours) can lead to irritation of the gums and ultimately a true infection represented by gum diseases such as gingivitis and periodontitis.

### SUMMARY OF THE INVENTION

Already for many years compounds have been tested for their ability to inhibit growth of cariogenic bacteria and to decrease the formation of dental plaque biofilms. None of these studies have lead to the successful development of products which can put a halt to the development of caries. Therefore a need still exists for potent agents, which can be used for the prevention or inhibition of progression of dental caries. Hence, it is an aspect of the invention to provide a novel alternative compound that preferably has a better functionality with respect to prevention and/or reducing (the risk of) dental caries.

It has surprisingly been found that certain small molecule inhibitors can be used to prevent maturation of dental plaque biofilms. It has also surprisingly been found that these inhibitors can be used to reduce the risk for oral diseases associated with functions that derive from a matured dental plaque bio film, including cariogenic activity (lactate production) or stimulation of gingival inflammation and infection.

In view of such use, the present invention also relates to oral compositions comprising these small molecule inhibitors.

It is well known that the great majority of the bacteria contributing to the formation of dental plaque bio films are Gram-positive bacteria. In particular, the bacteria that contribute to the formation of caries by production of lactic acid are known to be Gram-positive bacteria. This may be caused by the presence of fermentable sugars in the diet which favour these Gram-positive saccharolytic species in dental plaque. It is therefore very surprising to find that treatment of dental plaques with the small molecule inhibitors according to the present invention results in a pronounced reduction of lactic acid in dental plaque biofilms, even in the presence of fermentable sugars. Consequently, the present invention leads to a pronounced reduction of the risk for diseases associated with functions that derive from a matured dental plaque biofilm, such as dental caries.

### DETAILED DESCRIPTION OF THE INVENTION

According to one embodiment, the present invention relates to the use of a small molecule inhibitor for the manufacture of a medicament for the prevention of maturation of dental plaque biofilms and/or the reduction of the risk for diseases associated with functions that derive from matured dental plaque biofilm, wherein the small molecule inhibitor is characterized by the general Formula 1

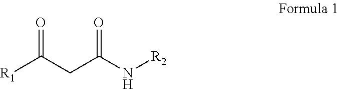

wherein R₁ can be a branched or unbranched hydrocarbon with three to nine carbon atoms, and

wherein R₂ can especially be a structure selected from the group consisting of

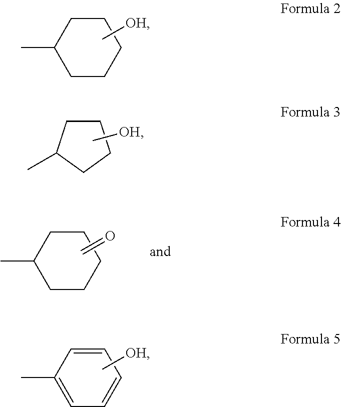

wherein each of the structures of Formula 2-5 may be further derivatized by at least one lower alkyl group and/or at least one halogen group.

According to a further embodiment, the present invention relates to the use of a small molecule inhibitor for the manufacture of a medicament for the prevention of maturation of dental plaque biofilms and/or the reduction of the risk for diseases associated with functions that derive from matured dental plaque biofilm, wherein the small molecule inhibitor is characterized by the general Formula 1

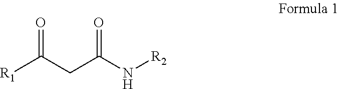

wherein R₁ can be a branched or unbranched hydrocarbon with especially three to nine carbon atoms, and

wherein R₂ can especially be a structure selected from the group consisting of

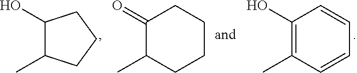

Diseases associated with functions that derive from matured dental plaque biofilm are for example gingivitis, periodontitis, caries, (apical) endodontitis, (peri)implantitis and halitosis.

In a further embodiment the invention relates to the small molecule inhibitor of Formula 1, especially for use in the prevention of maturation of dental biofilms and/or the reduction of the risk for diseases associated with functions that derive from matured dental plaque biofilm.

In a further embodiment the invention relates to the use of an effective amount of the small molecule inhibitor according to the present invention in a cosmetic product for the prevention of maturation of dental plaque biofilms.

In a further embodiment the present invention relates to the non-therapeutic use (such as the cosmetic use) of an effective amount of the small molecule inhibitor of Formula 1 for the prevention of maturation of dental plaque biofilms and for the reduction of the risk for diseases associated with functions that derive from matured dental plaque biofilm, including cariogenic activity (lactate production) or stimulation of gingival inflammation and infection (such as gingivitis and periodontitis).

In yet another embodiment, the invention also relates to a therapeutic use or therapeutic method in humans and/or animals using an effective amount of the small molecule inhibitor of Formula 1 for the prevention of maturation of dental plaque biofilms and/or for the reduction of the risk for diseases associated with functions that derive from an matured dental plaque biofilm, including cariogenic activity (lactate production) or stimulation of gingival inflammation and infection.

In a further embodiment the present invention relates to the use of an effective amount of the small molecule inhibitor of Formula 1 in a medicament or cosmetic product for the reduction of lactate production in dental biofilms.

In a further aspect the present invention relates to the use in the manufacture of a medicament or for non-therapeutic purposes of and effective amount of a compound of Formula 1 as further defined above for the inhibition of lactate production in dental plaque biofilms.

In a further aspect the present invention relates to the use in the manufacture of a medicament or for non-therapeutic purposes of an effective amount of a compound of Formula 1 as further defined above for the prevention or treatment of gingival inflammation or infection.

In a further embodiment the present invention relates to a compound of general Formula 1 wherein R₁ can be a branched or unbranched hydrocarbon with three to nine carbon atoms, and

wherein R₂ can be a structure especially selected from the group consisting of

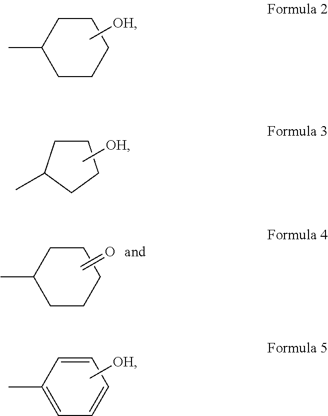

wherein each of the structures of Formula 2-5 may be further derivatized by at least one lower alkyl group and/or at least one halogen group, for use as a medicament.

In a further embodiment the present invention relates to a compound of general Formula 1 wherein R₁ can be a branched or unbranched hydrocarbon with three to nine carbon atoms, and

wherein R₂ can be a structure especially selected from the group consisting of

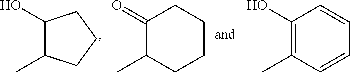

for use as a medicament.

In a particular embodiment R₁ in the compound of Formula 1 is an unbranched hydrocarbon with three to nine carbon atoms.

In a further particular embodiment R₂ in the compound of Formula 1 is

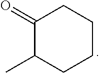

In a further particular embodiment, in the compound of Formula 1 R₁ is an unbranched hydrocarbon with three to nine carbon atoms and R₂ is

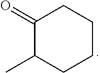

Compounds according to general Formula 1, wherein R1 is an unbranched nonyl group and R₂ is

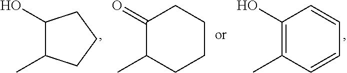

were reported to have antagonistic activity against quorum sensing of the Gram-negative bacteria Pseudomonas aeruginosa (Smith et al. (January 2003); Smith et al. (June 2003)).

For use according to the present invention the small molecule inhibitor of Formula 1 is presented to the dental plaque biofilms at a concentration of between 1 and 1000 μM. This concentration is considered an effective amount (an amount of the active compound which results in a local concentration of the active compound in the buccal cavity; see also below). A lower concentration may not be effective, while a higher concentration may lead to undesired side effects.

For the above use, the small molecule inhibitor of Formula 1 can be part of an oral composition.

The term “oral composition” as used herein refers to any composition, which can be introduced into the oral cavity and can be in contact with teeth, other than foods and drinks. The oral composition comprises the compound. The term “compound” may also refer to a combination of two or more different compounds of the type described herein (such as according to Formula 1). The oral composition can e.g. be a liquid (including a dispersion) or may be a paste. In a preferred embodiment, the oral composition may be non-viscous and may be a pourable liquid.

The term “oral composition” may refer to a cosmetic product, a drug or a quasi-drug or another composition. For example, the term “oral composition” may further refer to a cosmetic (more particularly, a dentifrice) which may have the effects of one or more of preventing tooth decay, whitening teeth, removing dental plaque, cleansing the oral cavity, preventing halitosis, removing tartar, preventing deposition of dental calculus, etc.

Specifically, the term “oral composition” of the present invention may refer to, for example, a dentifrice, a mouth wash (which can be applied by rinsing or using a device such as an oral irrigator), a troche, a gargle, a gum massage cream, a dental lozenge, artificial saliva, a dentifrice powder, granules, or a disintegrable tablet, a gel, a varnish, a toothpaste, a prophylaxis paste, a chewing gum, and a dry mouth paste. Hence, the oral composition may e.g. be a liquid, a paste or a chewing gum. In a further aspect, the invention also provide a container containing the oral composition.

The oral composition of this invention may also contain one or more of the following ingredients:
a polyol humectant, especially 5 to 90% by weight of one or more polyol humectants, such as glycerol, erythritol, sorbitol, mannitol, maltitol, xylitol or polyethylene glycol;
a sweetener, especially 0.001 to 5% by weight of one or more sweeteners, such as acesulfame and its salts, aspartame, dihydrochalcones, glycyrrhizin, glycyrrhizin derivative, licorice, saccharin, stevia, rebaudosides, sucralose, talin or thaumatins;
a mild abrasive, especially 1 to 60% by weight of one or more mild abrasives, having a hardness less than or equal to that of tooth enamel, such as calcium carbonate, dibasic calcium phosphate, tribasic calcium phosphate, calcium pyrophosphate, silica or hydroxyapatite;
a strong abrasive, especially 1 to 60% by weight of one or more strong abrasives, having a hardness greater than that of tooth enamel, such alumina, silica, titania, or fluoroapatite;
a flavor, especially 0.1 to 10% by weight of one or more flavor;
a surface active compound, especially 1 to 5% of one or more surface active compounds, such as sodium lauryl sulphate (SLS);
a fluoride containing compound, especially 1 to 5000 ppm by weight of one or more fluoride containing compounds, such as sodium fluoride, SnF₂, amine-fluoride or sodium mono fluorophosphate;
a mono-, di-, or polydentate acid or its salt, especially 0.1 to 10% by weight of one or more of mono-, di-, or polydentate acids or its salts such as citric acid, ethylenediaminetetraacetic acid, ascorbic acid, phosphoric acid, hydrochloric acid or sulfuric acid, to adjust and maintain the pH between 6 and 10;
a preservative, especially 0.1 to 1.0% by weight of one or more preservatives such as paraben, potassium sorbate, lactoferrin, lysozyme, or calcium propionate;
an antioxidant, especially 0.1 to 1.0% by weight of one or more antioxidants such as ascorbic acid, alpha-tocopherol, beta-carotene, coenzyme Q₁₀ or melatonin;
a thickener, especially 0.1 to 10% by weight of one or more thickeners such as colloidal cellulose, hydrated silica, polyethylene glycol, or polyvinylpyrrolidone;
5 to 95% by weight of water.

The above ingredients are preferably selected for compatibility with oral use and in particular should not comprise ingredients with toxic or otherwise undesirable side effects upon application to the oral cavity or upon ingestion.

The oral compositions may comprise the compound of general Formula 1 in an amount of 1 to 10,000 μM, relative to the total weight of the oral composition. Smaller amounts may not be effective during normal use, while larger amounts may have an undesired effect and may optionally effect the texture and/or flavor of the oral composition. With such oral composition, one may provide for instance the above indicated concentration of 1 and 1000 μM in the buccal cavity when applying the oral composition to this cavity.

Accordingly in a further aspect, the present invention relates to an oral composition comprising an effective amount of a compound of general Formula 1 wherein R₁ can be a branched or unbranched hydrocarbon with three to nine carbon atoms, and

wherein R₂ can be a structure especially selected from the group consisting of

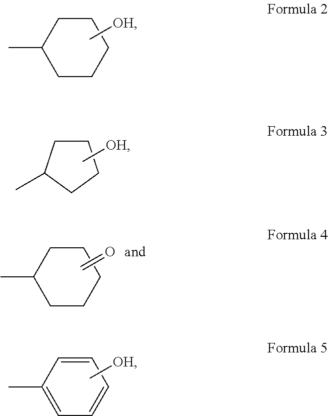

wherein each of the structures of Formula 2-5 may be further derivatized by at least one lower alkyl group and/or at least one halogen group

In a further aspect, the present invention relates to an oral composition comprising an effective amount of a compound of general Formula 1 wherein R₁ can be a branched or unbranched hydrocarbon with three to nine carbon atoms, and wherein R₂ can be a structure especially selected from the group consisting of

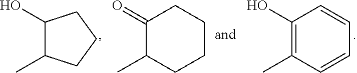

In a particular embodiment, the present invention relates to an oral composition comprising a compound of general Formula 1, wherein R₁ is an unbranched hydrocarbon with three to nine carbon atoms.

In a further particular embodiment, the present invention relates to an oral composition comprising an effective amount of a compound of general Formula 1, wherein R₂ is

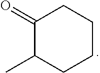

In a further embodiment, the present invention relates to an oral composition comprising an effective amount of a compound of general Formula 1 wherein R₁ is an unbranched hydrocarbon chain of nine carbon atoms and R₂ is

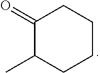

As indicated above oral compositions of the present invention can be applied using a suitable device, for example an oral irrigator, such as an airfloss or a waterfloss, such as the devices e.g. described in WO2012117313 and in U.S. Pat. No. 4,302,186, which are incorporated herein by reference. Therefore, according to a further aspect, the invention relates to a device, especially an electronic device, such as a powered toothbrush, an interdental cleaner, an oral irrigator, a tongue scraper, or any other appropriate intra-oral delivery system, for application of oral compositions further comprising an oral composition comprising an effective amount of a small molecule inhibitor of Formula 1. The device may especially be configured to host a replaceable or refillable container. Such container can be replaced, when empty, with a new full container. Or, alternatively or additionally, the device comprises a refillable container, that can be refilled when empty.

A therapeutic or cosmetic use or therapeutic method of the compounds and compositions of the present invention also comprises the use of these compound or compositions together with a suitable device, for example an oral irrigator, such as an airfloss or a waterfloss as referred above.

The term “in an effective amount” as used herein refers to an amount of the active compound which results in a local concentration of the active compound in the buccal cavity and in particular at the location of the teeth, which is high enough to establish the desired effect of prevention of maturation of dental plaque bio films and reduction of the risk for diseases associated with functions that derive from a matured dental plaque biofilm, including cariogenic activity (lactate production) or stimulation of gingival inflammation and infection (e.g. gingivitis and periodontitis).

Surprisingly, it has been found that the concentration of the small molecule inhibitor of Formula 1 suitable to establish the desired effect of prevention of maturation of dental plaque bio films and reduction of the risk for diseases associated with functions that derive from an matured dental plaque biofilm can be lower than the inhibitory concentration.

The term “inhibitory concentration” as used herein refers to a concentration that completely inhibits oral plaque bio film growth.

For example, for the compound of general Formula 1 wherein R₁ is an unbranched hydrocarbon chain of nine carbon atoms and R₂ is

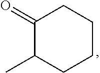

the desired effect can be observed at a concentration of between 10 μM and 100 μM, whereas the inhibitory concentration of this compound is above 800 μM.

The compounds of general Formula 1 can be prepared according to the methods described in Smith et al. (January 2003) and Smith et al. (June 2003) or by methods analogous thereto.

Further, especially the groups R2 as defined herein, even more especially as defined in the independent claims, are not further derivatized (by at least one lower alkyl group and/or at least one halogen group), though in other embodiments derivatization is not excluded. Further, R1 may especially be selected from propyl, butyl, pentyl, hexyl, octyl and nonyl. In yet other aspects, R1 may be selected from ethyl and propyl. In yet another aspect, R1 may be H. Especially, R1 is a branched or unbranched hydrocarbon with three to nine carbon atoms. A lower alkyl is especially a an alkyl having up to 4 carbon atoms.

The term “small molecule inhibitor” especially refers to a low molecular weight compound suitable to prevent maturation of dental plaque bio films and/or to reduce of the risk for diseases associated with functions that derive from matured dental plaque biofilm, and in particular to reduce the production of lactic acid by matured dental plaque bio films. Instead of the term “small molecule inhibitor” optionally also the term “compound” may be applied.

The term “comprise” includes also embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

### BRIEF DESCRIPTION OF THE DRAWINGS

FIG. 1 shows the in vitro oral plaque bio film formation expressed as colony forming units (CFU). The concentrations of the compounds are shown on the x-axis. Statistically significant differences in comparison with the control are marked with an asterisk (*).

FIG. 2 shows total lactic acid production for biofilms grown in the presence of homoserinelactone molecules with different C-chain lengths. Also 3-Oxo-N(2-oxocyclohexyl)dodecanamide was tested, and DMSO is used as control. 100 μM of each compound is used. Lactic acid production is given per biofilm after 3 h incubation in buffered peptone water (BPW) containing 0.2% sucrose. Significance compared to the control is shown: *** P<0.01.

FIG. 3 shows total lactic acid production for biofilms grown in the presence of different concentrations 3-Oxo-N(2-oxocyclohexyl)dodecanamide and HSL, in mM per biofilm after 3 h incubation in BPW containing 0.2% sucrose. All concentrations of 3-Oxo-N(2-oxocyclohexyl)dodecanamine result in a statistical significant difference compared to the control.

FIG. 4A shows lactate production in mM lactate; and FIG. 4B shows lactate production corrected for CFU, expressed in μM lactate/1×10⁶ CFU. Concentrations of the compounds are shown on the x-axis. Statistically significant differences in comparison with the control samples are marked with an asterisk (*).

FIG. 5 shows the compositional shift of in vitro oral plaque biofilms (A %) with respect to the most abundant species (Streptococcus (A) and Veillonella (B)) grown after 48 and 96 hours upon addition of 100 μM 3-oxo-N(2-oxocyclohexyl)dodecanamide (a), 10 μM Furanone C30 (b) or 10 μM 3,4-Dibromo-2(5H)-furanone (c), compared to the control biofilms.

### EXAMPLES

### Materials & Methods

### Inoculum Collection

Stimulated saliva, used as inoculum, was collected on ice from ten donors. Saliva was donated 24 h after last brushing. The saliva was diluted 2-fold with 60% sterile glycerol, aliquoted and stored at −80° C. Before use, a pooled sample was prepared by mixing 200 μl of thawed saliva of each donor and vortexing for 30 seconds. In vitro oral plaque biofilms were inoculated 1:50 with pooled saliva.

### Test Compounds

All compounds tested of Table 1 were obtained from Sigma Aldrich. All compounds of Table 2, except 3-oxo-N(2oxocyclohexyl)dodecanamide, were kindly provided by M. Meijler, University of the Negev, Israel. The compounds were dissolved in DMSO and stored at −20 C. The compounds were continuously present at the indicated concentrations during biofilm growth, but not during further phenotypic analysis, i.e. lactate production.

TABLE 1
Compounds tested in in vitro oral plaque biofilm model (Example 1)

| | | Concentration | |
|---|---|---|---|
| | Compound | used (in μM) | Reference |
| 3-Oxo-N | 3-oxo-N-(2- | 0.01-100 | Smith et al. |
| | oxocyclohexyl)dodecanamide | | (Jan. 2003) |
| 3,4- | 3,4-Dibromo-2(5H)-furanone | 10 | |
| dibromo | | | |
| Furanone | (Z-)-4-Bromo-5- | 10 | Wu et al. |
| C30 | (bromomethylene)- | | (2004) |
| | 2(5H)-furanone | | |

TABLE 2
Compounds tested for effect on lactic acid production (Example 2)

| | Compound |
|---|---|
| HSL-C6 | N-[(3S)-tetrahydro-2-oxo-3-furanyl]-hexanamide |
| HSL-C7 | N-[(3S)-tetrahydro-2-oxo-3-furanyl]-heptanamide |
| HSL-C8 | N-[(3S)-tetrahydro-2-oxo-3-furanyl]-octanamide |
| HSL-C9 | N-[(3S)-tetrahydro-2-oxo-3-furanyl]-nonanamide |
| HSL-C10 | N-[(3S)-tetrahydro-2-oxo-3-furanyl]-decanamide |
| HSL-C11 | N-[(3S)-tetrahydro-2-oxo-3-furanyl]-undecanamide |
| HSL-C12S | N-[(3S)-tetrahydro-2-oxo-3-furanyl]-dodecanamide |
| HSL-C12R | N-[(3R)-tetrahydro-2-oxo-3-furanyl]-dodecanamide |
| HSL-C13 | N-[(3S)-tetrahydro-2-oxo-3-furanyl]-tridecanamide |
| 3-Oxo-N | 3-oxo-N-(2-oxocyclohexyl)dodecanamide |

### In Vitro Oral Plaque Biofilm Formation

In vitro oral plaque biofilms were grown in the Amsterdam Active Attachment Model (AAA-model, Exterkate et al. (2010)), assembled with glass coverslips (diameter 12 mm; Menzel, Braunschweig, Germany). The model was inoculated for 8 h with pooled saliva in buffered semi-defined McBain medium (2.5 g/l mucin (Sigma, St Louis, Mo.), 2.0 g/l Bacto peptone (Difco, Detroit, Mich.), 2.0 g/l Trypticase peptone (BBL, Cockeysville, Md.), 1.0 g/l yeast extract (BD Diagnostic Systems, Sparks, Md.), 0.35 g/l NaCl, 0.2 g/l KCl, 0.2 g/l CaCl₂, 1 mg/l hemin (Sigma, St. Louis, Mo., USA), and 2 mg/l vitamin K1 (McBain et al. (2005)), with 50 mmol/1 PIPES at pH 7.0), with 0.2% sucrose.

In vitro oral plaque biofilms were grown in the AAA-model for 48-96 h in the presence of the compounds tested. The model was inoculated with saliva and incubated anaerobically at 37° C. for 8 hours to allow microbes to attach to the glass coverslips. After 8 hours of attachment, the inoculation medium was refreshed and in vitro oral plaque biofilms were grown for 16 hours. This refreshment routine was repeated daily until the day of harvesting.

In vitro oral plaque biofilms were harvested by transferring the glass coverslips into 2 ml phosphate buffered saline (PBS). The biofilms were dispersed using a Vibracell VCX130 sonicator with a maximum of 130 Watts and 20 kHz (Sonics & Materials, Newtown, USA). Bio films were sonicated on ice for 1 minute with a pulse rate of 50% and pulses of 1 second. Vibration amplitude was set to 40%.

### CFU Determination

To estimate the amount of in vitro oral plaque biofilm formation, total anaerobic colony forming units were determined. Briefly, serial dilutions of the dispersed bio films were made and plated on tryptic soy agar blood plates. Plates were subsequently incubated anaerobically for 96 h at 37° C. and colony forming units were determined by counting the number of colonies for each dilution.

### Lactic Acid Production Assay

To estimate the cariogenic phenotype, lactic acid production of the in vitro oral plaque biofilms was determined prior to harvesting (Exerkate et al 2010). In short, the biofilms on coverslips were placed in a 24-well plate containing 1.5 ml of BPW with 0.2% sucrose. Lactic acid formation was allowed for 3 hr at 37° C. under anaerobic conditions. The total amount of lactic acid produced in this period was analyzed using a colorimetric assay described previously (van Loveren et al. (2000)) and expressed as mM lactic acid, and as μM lactic acid per 1×10⁶ CFU.

### DNA Isolation and PCR

DNA was isolated using phenol bead-beating followed by Agowa isolation, following the manufacturers instructions (LGC Genomics, Mag mini kit). Briefly, cells were mechanically disrupted four times at 1200 rpm for two minutes in addition of Tris-saturated Phenol, pH8, 0.1 mm zirconium beads and Mag lysis buffer. The DNA-containing phase was mixed with binding buffer and magnetic beads. DNA concentration was estimated by determining the absorbance at 260 nm using the nanodrop (Isogen, ND-1000).

The primers used for 16S rDNA PCR amplification are listed in Table 2. PCR was performed in a final volume of 25 μl containing 1 μl of each primer (10 mM), 1 μl dNTP (10 mM), 2.5 μl 10× DreamTaq Buffer including MgCl₂ (Thermo Scientific), 0.2 μl DreamTaq DNA Polymerase (5 u/μ1, Thermo Scientific) and 50 ng of DNA. Initial denaturation was performed at 94° C. for 4 min, followed by 35 cycles of denaturation at 94° C. for 30 s, 54° C. annealing for 1 min, 72° C. for 1 min primer extension and a final extension at 72° C. for 5 min. Product formation was confirmed by electrophoresis of 5 μl on a 1% (w/v) agarose gel (Sphearo Q, Leiden, The Netherlands) stained with ethidium bromide.

These samples were analyzed by 16S rDNA Illumina sequencing at TNO Zeist (Netherlands).

### Example 1

### Testing of Compounds of Table 1 in Amsterdam Active Attachment Model

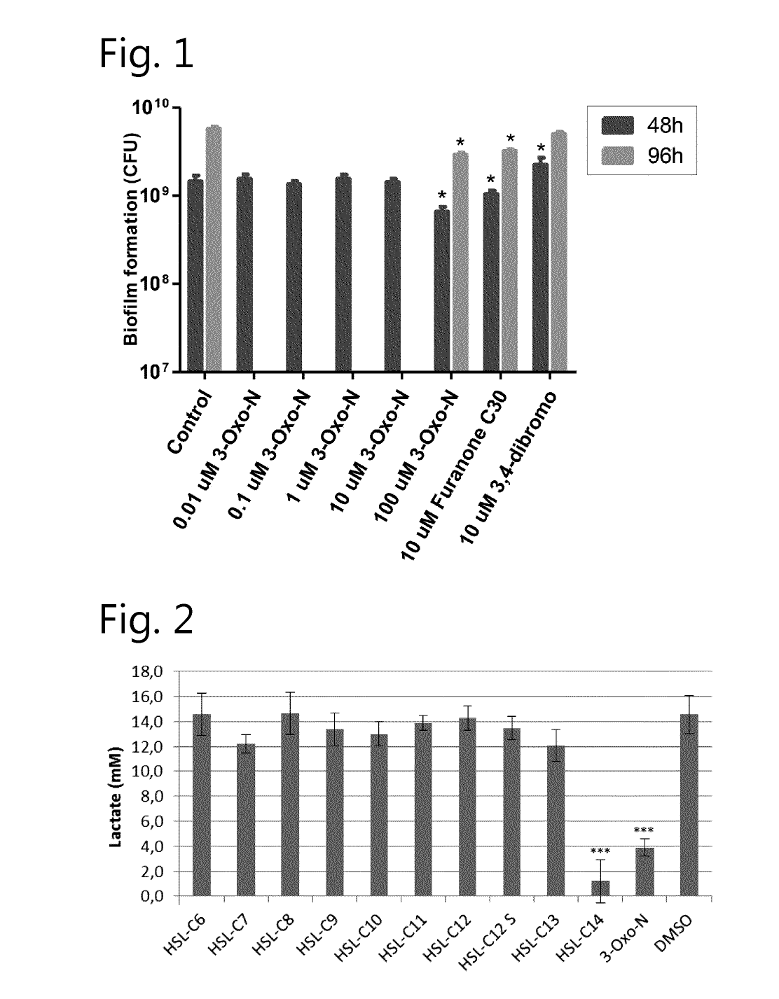

In vitro oral plaque biofilm formation was slightly affected by 3-Oxo-N-(2-oxocyclohexyl)dodecanamide, but only for the highest concentration tested (100 μM). With 100 μM of this compound present, in vitro oral plaque biofilms established up to 6.6×10⁸ Colony Forming Units (CFU) versus 1.5×10⁹ CFU for the control and all the other concentrations (FIG. 1). Furanone C30 and 3,4-dibromo-2(5H)-furanone also slightly effect in vitro oral plaque biofilm formation but since the difference is less than one log, this is not considered biologically relevant.

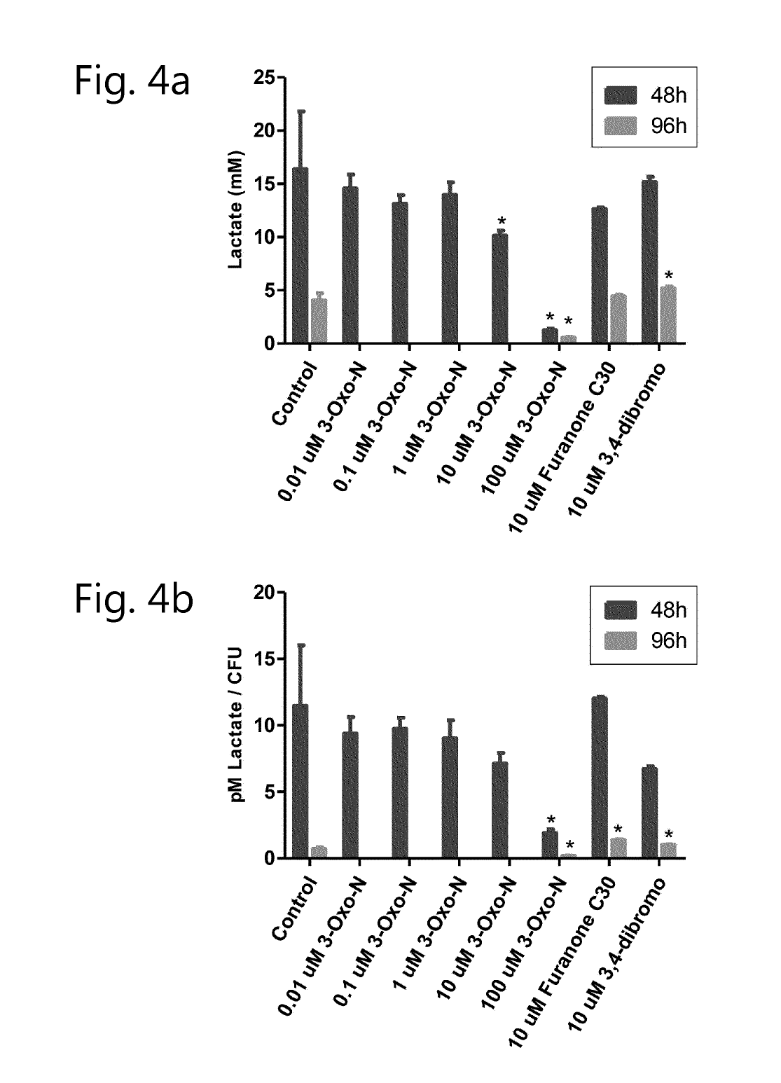

Caries is related to high lactate production by dental plaque biofilms. Therefore, lactate production was measured for all in vitro oral plaque biofilms. FIG. 4A shows that lactate production is almost completely blocked by the addition of 100 μM of 3-oxo-N(2-oxocyclohexyl)dodecanamide. Addition of 10 μM of this compound also significantly reduces lactate production of the in vitro oral plaque biofilm. Although lactate production of the control in vitro oral plaque biofilm was lower after 96 hours of growth than after 48 hours of growth, the reducing effect was still present. The highest concentration of 3-Oxo-N(2-oxocyclohexyl)dodecanamide also slightly reduced total biomass, but this cannot explain the lower lactate production. FIG. 4B shows that when corrected for CFU, lactate production is still lower upon addition of 100 μM or 10 μM of this compound.

The reduction of lactate production is not observed for furanone C30 and for 3,4-dibromo-2(5H)-furanone. After 96 h of growth both furanones even seem to up-regulate the lactic acid production of the in vitro oral plaque biofilms.

### Example 2

### Lactic Acid Production in Presence of C12-HSL and Structural Variants Thereof

Lactate production of biofilms grown in the presence of homoserine lactone (HSL) molecules with different C-chains (compounds of Table 2) was measured. 3-Oxo-N-(2-oxocyclohexyl)dodecanamide has a C12 chain, so the HSL-C12 QS molecule is the most similar structurally. HSL-C12(S) is the active form of the QS molecule, HSL-C12(R) is the inactive enantiomer. For the C12-chain, both enantiomers were tested.

Biofilms were grown for 48 h in buffered McBain medium supplemented with 0.2% sucrose in the presence of 100 μM of one of the compounds. Medium was refreshed twice every day. After 48 h of growth, biofilms were transferred to BPW with 0.2% sucrose, and biofilms were allowed to produce lactate for 3 h.

FIG. 2 shows the lactate production of the biofilms. Only HSL-C14 and 3-Oxo-N show a reduced lactic acid production. For HSL-C14, this can be explained by a lack of biofilm production as the compound is toxic to the formation of biofilm. This was not the case for 3-oxo-N, were normal amounts of biofilm were visible.

The data obtained show no effect of the natural homoserine lactone (C12-HSL) or its structural variants on lactic acid production. This indicates that it is not likely that 3-Oxo-N-(2-oxocyclohexyl)dodecanamide acts as a inhibitor of QS. The observed effect therefore has to be related to a currently unknown effect of 3-Oxo-N-(2-oxocyclohexyl)dodecanamide on the composition and behavior of in vitro oral dental plaque.

### Example 3

No Interference of the Quorum Sensing Natural Homoserine Lactone with 3-Oxo-N-(2-Oxocyclohexyl)Dodecanamide

In this experiment the effect of the quorum sensing molecule C12-HSL on the lactate reducing activity of 3-Oxo-N-(2-oxocyclohexyl)dodecanamide was tested. Biofilms were grown for 48 h in buffered McBain medium supplemented with 0.2% sucrose, in the presence of 0, or 25 or 100 μM 3-oxo-N(2-oxocyclohexyl)dodecanamide and 0, or 25 or 100 μM of C12-HSL. Medium was refreshed twice every day. After 48 h of growth, biofilms were transferred to BPW with 0.2% sucrose, and biofilms were allowed to produce lactate for 3 h.

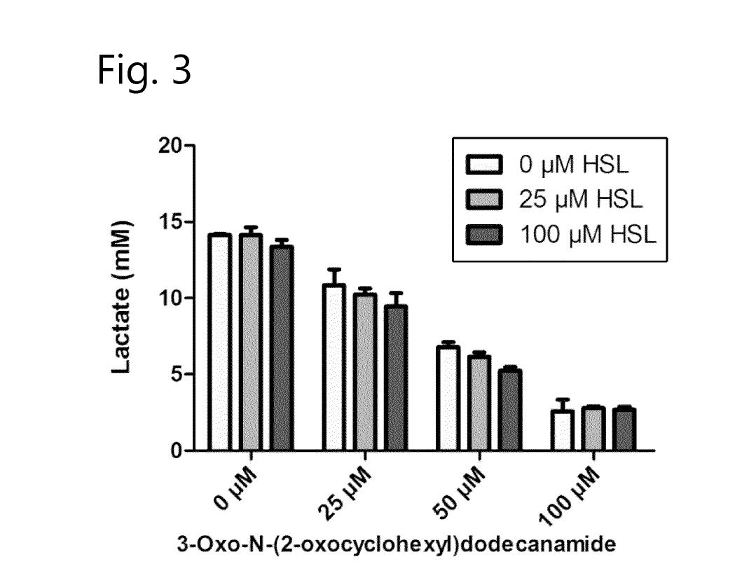

FIG. 3 shows the total lactic acid production per bio film. It can concluded that the QS molecule does not reduce the effect of our compound. This points towards mechanism for the reduction of lactate production by the compounds of the present invention different from quorum sensing.

### Example 4

### In Vivo Oral Plaque Biofilm Composition in the Presence of Quorum Sensing Inhibitors

The composition of the biofilms is studied using 16S rDNA sequencing (Tables 4A and 4B). For all bio films, Veillonella and Streptococcus are the two dominating genera, but their ratio is different. After 48 h, a shift in composition is observed for the biofilms grown with addition of 100 μM 3-Oxo-N(2-oxocyclohexyl)dodecanamide in comparison with the control. Veillonella, known to consume lactate, is 10% more abundant in biofilms grown with addition of this compound and Streptococcus is more than 30% reduced in these biofilms (Table 2). The decrease in Streptococcus spp in the 3-Oxo-N(2-oxocyclohexyl)dodecanamide grown biofilms is accompanied with an increase in Actinobacillus species. For both furanone C30 and 3,4-dibromo-2(5H)-furanone this effect is absent or even inversed.

All 96 h biofilms grown with addition of a quorum sensing inhibitor show a decrease in Streptococcus and an increase in Veillonella and Actinobacillus (Table 2).

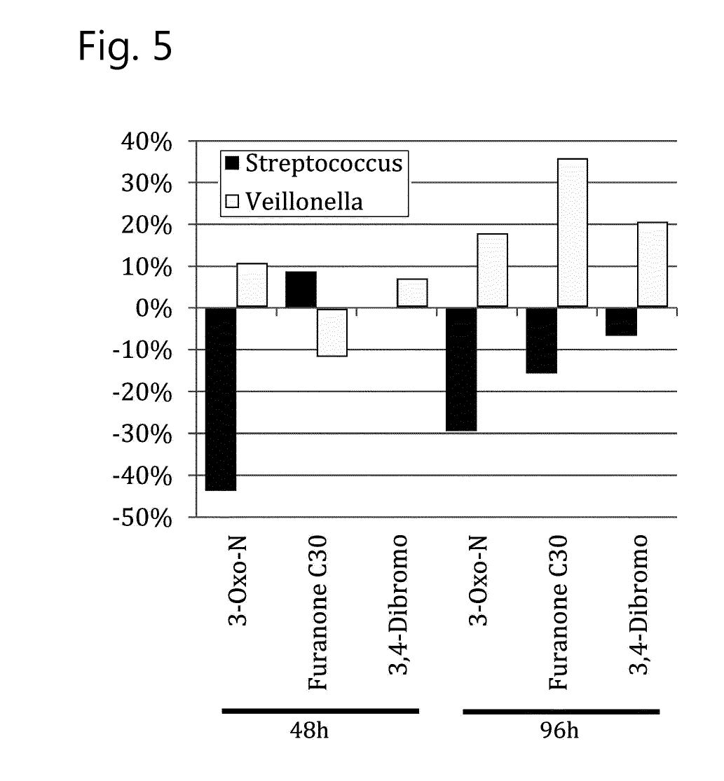

The compositional shift of biofilms (A %) with respect to the most abundant species (Streptococcus (A) and Veillonella (B)) grown after 48 and 96 hours is shown in FIG. 5.

Table 3. Microbial composition of biofilms grown for 48 (Table 3A) or 96 h (Table 3B) in the presence of different QS inhibitors. Composition was determined by 16S rDNA sequencing. The composition of the original inoculum used for all biofilms was also determined. “Unclass.” means: unclassified.

TABLE 3A

| | 48 h | | | | |
|---|---|---|---|---|---|
| taxon | Control | 3-Oxo-N | Furanone C30 | 3,4-Di-bromo | Inoculum |
| Streptococcus | 60.8% | 34.3% | 66.2% | 60.8% | 19.5% |
| Veillonella | 32.5% | 36.0% | 28.6% | 34.8% | 23.4% |
| Actinobacillus | 5.3% | 27.6% | 4.9% | 3.7% | 15.0% |
| Prevotella | 0.3% | 0.0% | 0.0% | 0.0% | 16.8% |
| Campylobacter | 0.3% | 1.1% | 0.0% | 0.2% | 1.1% |
| Neisseria | 0.0% | 0.0% | 0.0% | 0.0% | 6.4% |
| Megasphaera | 0.0% | 0.0% | 0.0% | 0.0% | 1.2% |
| Porphyromonas | 0.0% | 0.0% | 0.0% | 0.0% | 2.7% |
| Solobacterium | 0.1% | 0.0% | 0.1% | 0.1% | 0.1% |
| Unclass. Pasteurellaceae | 0.4% | 0.6% | 0.0% | 0.2% | 0.5% |
| Fusobacterium | 0.0% | 0.0% | 0.0% | 0.0% | 2.4% |
| Granulicatella | 0.1% | 0.0% | 0.0% | 0.1% | 0.8% |
| Haemophilus | 0.1% | 0.2% | 0.0% | 0.0% | 0.9% |
| Actinomyces | 0.0% | 0.0% | 0.1% | 0.0% | 0.8% |
| Rothia | 0.0% | 0.0% | 0.0% | 0.0% | 1.3% |
| Other | 0.1% | 0.1% | 0.0% | 0.1% | 7.2% |

TABLE 3B

| | 96 h | | | | |
|---|---|---|---|---|---|
| taxon | Control | 3-Oxo-N | Furanone C30 | 3,4-Di-bromo | Inoculum |
| Streptococcus | 40.7% | 28.7% | 34.4% | 38.0% | 19.5% |
| Veillonella | 43.8% | 51.7% | 59.6% | 52.9% | 23.4% |
| Actinobacillus | 2.1% | 12.0% | 4.5% | 3.4% | 15.0% |
| Prevotella | 3.8% | 0.0% | 0.0% | 2.3% | 16.8% |
| Campylobacter | 3.7% | 7.2% | 0.0% | 1.6% | 1.1% |
| Neisseria | 0.0% | 0.0% | 0.0% | 0.0% | 6.4% |
| Megasphaera | 2.8% | 0.0% | 0.0% | 0.0% | 1.2% |
| Porphyromonas | 0.0% | 0.0% | 0.0% | 0.0% | 2.7% |
| Solobacterium | 0.6% | 0.0% | 0.8% | 0.7% | 0.1% |
| Unclass. Pasteurellaceae | 0.3% | 0.0% | 0.0% | 0.0% | 0.5% |
| Fusobacterium | 0.2% | 0.0% | 0.0% | 0.0% | 2.4% |
| Granulicatella | 0.1% | 0.0% | 0.3% | 0.2% | 0.8% |
| Haemophilus | 0.0% | 0.1% | 0.0% | 0.0% | 0.9% |
| Actinomyces | 0.1% | 0.0% | 0.3% | 0.1% | 0.8% |
| Rothia | 0.0% | 0.0% | 0.0% | 0.0% | 1.3% |
| Other | 1.7% | 0.3% | 0.1% | 0.7% | 7.2% |

### Example 5

### Toothpaste

Toothpaste according to the present invention may contain the following ingredients (in weight/weight %):
20-50% abbrassive;
20-30% humectant such as sorbitol;
1-5% surface-active compound; 1-5% thickener; 1-2% flavor compound; 0.5-2% white dye compound; 0.05-0.5% preservative; 0.5-5% 3-oxo-N-(2-oxocyclohexyl)dodecanamide
20-30% water

### REFERENCES CITED

1) Smith et al. (January 2003): Smith, K. M., Bu, Y. and Suga, H. “Induction and inhibition of Pseudomonas aeruginosa quorum sensing by synthetic autoinducer analogs” Chem. Biol. 2003 January; 10(6): 81-9
2) Smith et al. (June 2003): Smith, K. M., Bu, Y. and Suga, H. “Library screening for synthetic agonists and antagonists of a Pseudomonas aeruginosa autoinducer” Chem. Biol. 2003 June; 10(6): 563-71.
3) Wu et al. (2004): Wu, H., Song, Z., Hentzer, M., Andersen, J. B., Molin, s., Givskov, M. and Hoiby, N. “Synthetic furanones inhibit quorum-sensing and enhanced bacterial clearance in Pseudomonas aeruginosa lung infection in mice” J. Antimicrob. Chemother. 2004, 53, 1054-61.
4) Exterkate et al. (2010): R. A. M. Exterkate, W. Crielaard, J. M. Ten Cate “Different response to amine fluoride by Streptococcus mutans and polymicrobial bio films in a novel hight-throughput active attachment model”; Caries Res. 2010; 44:372-379
5) McBain et al. (2005): A. J. McBain, C. Sissons, R. G. Ledder, P. K. Sreenivasan, W. De Vizio, P. Gilbert “Development and characterization of a simple perfused oral microcosm” J. Appl. Microbiol. 2005, 98, 624-634
6) Van Loveren et al. (2000): van Loveren C., Buijs J. F., ten Cate J. M. “The effect of triclosan toothpaste on enamel demineralization in a bacterial demineralization model” J. Antimicrob. Chemother. 2000; 45:153-158.

1. A small molecule inhibitor for use in the prevention of maturation of dental plaque biofilms and/or the reduction of the risk for diseases associated with functions that derive from matured dental plaque biofilms selected from the group consisting of caries, gingival inflammation and infections such as gingivitis and periodontitis, wherein the small molecule inhibitor is characterized by the general Formula 1 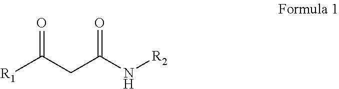
wherein R1 is an unbranched hydrocarbon with three to nine carbon atoms, and wherein R2 is

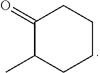

2.-4. (canceled)

5. A small molecule inhibitor according to claim 1, wherein the small molecule inhibitor is presented to the dental plaque biofilms at a concentration, which is sub-inhibitory.

6. A small molecule inhibitor according to claim 1 wherein the small molecule inhibitor is presented to the dental plaque biofilms at a concentration of between 1 and 1000 μM.

7. A small molecule inhibitor according to claim 1, wherein the small molecule inhibitor affects a reduction of lactate production in dental plaque biofilms.

8. Oral composition for use in the prevention of maturation of dental plaque biofilms and/or the reduction of the risk for diseases associated with functions that derive from matured dental plaque biofilms, selected from the group consisting of caries, gingival inflammation and infections such as gingivitis and periodontitis, comprising an effective amount of a compound of general Formula 1 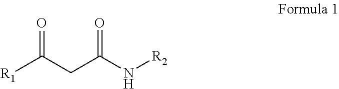
wherein R1 is an unbranched hydrocarbon with three to nine carbon atoms, and wherein R2 is

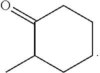

9.-11. (canceled)

12. Oral composition according to claim 8, which comprises the compound of general Formula 1 at a concentration of 1-10,000 μM.

13. Oral composition according to claim 8 wherein the oral composition is a liquid or a paste.

14. Oral composition according to claim 8, wherein the oral composition is a chewing gum.

15. An electronic device, such as a powered toothbrush, an interdental cleaner, an oral irrigator, a tongue scraper, or any other appropriate intra-oral delivery system, for application of an oral composition according to claim 8.
